(19) United States
(12) Patent Application Publication
(10) Pub. No.: US 2015/0297860 A1
(43) Pub. Date: Oct. 22, 2015

(54) THERAPEUTIC ANATOMICAL WRAP

(71) Applicant: ROGER J. KEALEY, Canton, GA (US)

(72) Inventor: ROGER J. KEALEY, Canton, GA (US)

(21) Appl. No.: 14/672,540

(22) Filed: Mar. 30, 2015

Related U.S. Application Data
(60) Provisional application No. 61/980,202, filed on Apr. 16, 2014.

(51) Int. Cl. A61M 21/02; A61F 7/02

(52) U.S. Cl. CPC A61M 21/02; A61F 7/02; A61M 2021/0016

(57) ABSTRACT

Disclosed are various embodiments of apparatuses and methods related to a therapeutic wrap that provides aromatherapy in addition to therapeutic relief to bodily discomfort due to aches and pains. The therapeutic wrap may comprise an elongated fabric comprising one or more sealed chambers filled with a natural substance mixture. A pocket may be attached to the therapeutic wrap that is sized to receive a removable insert scented with an aromatherapy fragrance or essential oil.

### CROSS REFERENCE TO RELATED APPLICATIONS

This application claims priority to copending U.S. Provisional Patent Application titled “MULTI-CHAMBER SCENTED THERAPEUTIC ANATOMICAL WRAP” filed on Apr. 16, 2014 and assigned application No. 61/980,202, which is incorporated by reference herein in its entirety.

### BACKGROUND

Aches and pains experienced by the body can be caused by a variety of reasons. Heated and cool wraps may be used for therapeutic relief from the aches and pains.

### SUMMARY

Included are apparatuses and methods for a therapeutic wrap. One embodiment of an apparatus, among others, includes a therapeutic wrap, comprising an inner wrap extending from a first end to a second end and comprising one or more sealed chambers having a natural substance for providing therapeutic relief; an outer sleeve surrounding a portion of the inner wrap; a pocket attached to the outer sleeve; and a scented insert inserted within the pocket, the scented insert being scented with an aromatherapy fragrance/essential oil.

Another embodiment of an apparatus, among others, includes a therapeutic wrap, comprising: an elongated fabric comprising one or more sealed chambers filled with a natural substance mixture; one or more pocket attached to the elongated fabric; and a scented insert inserted into one or more pockets, the scented insert being scented with an aromatherapy fragrance/essential oil.

Another embodiment of a method, among others, includes a method, comprising: adding an aromatherapy fragrance/essential oil to one or more removable inserts; inserting the one or more removable inserts into one or more pockets attached to a therapeutic wrap, wherein the therapeutic wrap comprises one or more natural substances used for therapeutic relief; positioning the therapeutic wrap about a portion of a body; and securing the therapeutic wrap about the portion of the body.

Other embodiments, systems, methods, features, and advantages of this disclosure will be or will become apparent to one with skill in the art upon examination of the following drawings and detailed description. It is intended that all such additional apparatuses, methods, features, and advantages be included within this description and be within the scope of the present disclosure.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing of an example of a therapeutic wrap in use according to various embodiments of the present disclosure.

FIG. 2 is a drawing of an example of an inner wrap of the therapeutic wrap of FIG. 1 according to various embodiments of the present disclosure.

FIG. 3 is a drawing of a multi-chambered filled insert of the inner wrap of FIG. 2 according to various embodiments of the present disclosure.

FIG. 4 is a drawing of an example of a front view of the therapeutic wrap of FIG. 1 where a scented insert is inserted into a pocket on the outer sleeve of the therapeutic wrap according to various embodiments of the present disclosure.

FIG. 5 is a drawing of an example of a back view of the therapeutic wrap of FIG. 1 showing a lack of pockets on the back of the therapeutic wrap according to various embodiments of the present disclosure.

FIG. 6 is a drawing of an example of a front view of an outer sleeve of the therapeutic wrap of FIG. 1 illustrating the pockets surrounding the ends of the outer sleeve according to various embodiments of the present disclosure.

FIG. 7 is a drawing of an example of a back view of the outer sleeve of FIG. 6 illustrating the pockets surrounding the ends of the outer sleeve according to various embodiments of the present disclosure.

FIG. 8 is drawing of an example of a front view of another embodiment of a therapeutic wrap where the therapeutic wrap is a one-piece wrap with pockets directly attached to the therapeutic wrap for receiving scented inserts.

FIG. 9 is a drawing of an example of a back view of the therapeutic wrap of FIG. 8 where a band is attached to the back of the therapeutic wrap used for securing placement of the wrap.

FIG. 10 is a drawing of an example of another embodiment of an inner wrap of the therapeutic wrap of FIG. 1 showing the one or more chambers as part of the inner wrap.

FIG. 11 is a flowchart illustrating methods according to various embodiments of the present disclosure.

### DETAILED DESCRIPTION

In the following discussion, a general description of apparatuses and methods according to various embodiments of the present disclosure is provided, followed by a discussion of the operation of the same. Embodiments of the present disclosure relate to a therapeutic wrap. More specifically, disclosed herein are novel approaches to the implementation of a therapeutic wrap that relieves bodily discomfort due to aches and pains and further provides the benefit of aromatherapy. The therapeutic wrap includes one or more pockets that are designed to receive removable inserts that are scented with fragrances and/or essential oils to provide the additional benefit of aromatherapy. In some embodiments, the inserts may be scented by applying one or more drops of essential and/or fragrant oil used for the purpose of altering a person's mood or behavior. In other embodiments, the therapeutic wrap may remain unscented.

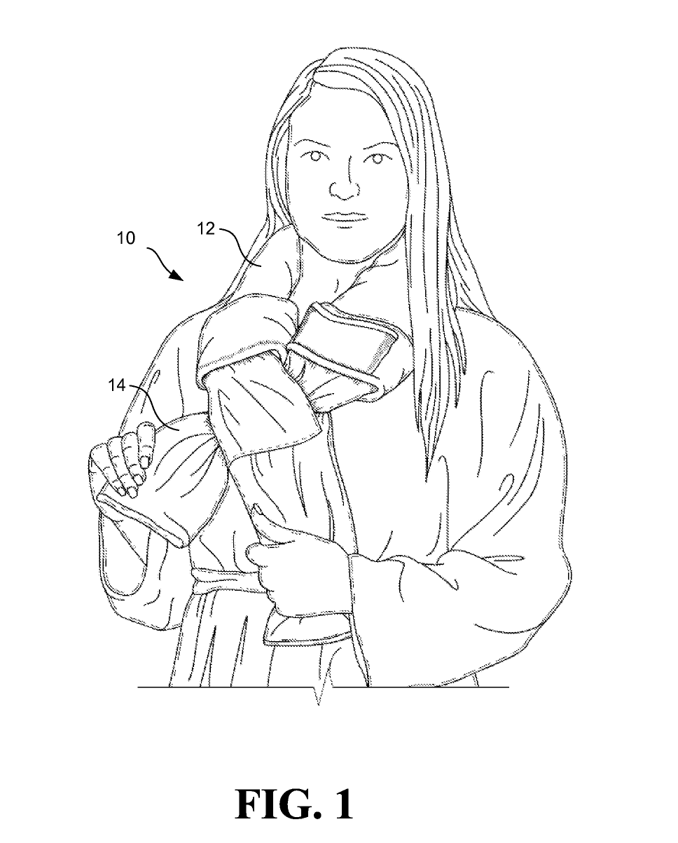

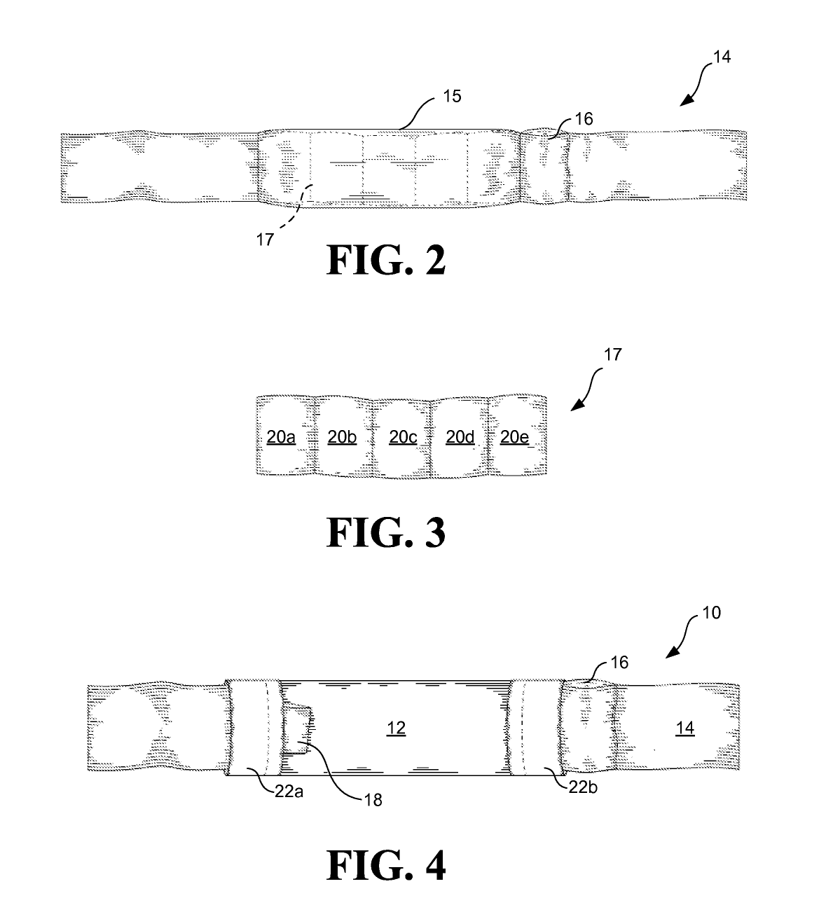

Referring now to FIG. 1, shown is a drawing of an example of a therapeutic wrap 10 being worn by a person according to various embodiments of the present disclosure. The therapeutic wrap 10 of FIG. 1 comprises a removable outer sleeve 12 surrounding a portion of an inner wrap 14. As will be described in greater detail below, the inner wrap 14 includes a middle region 15 (FIG. 2) that comprises natural grain-like substances for providing therapeutic relief. The inner wrap 14 further comprises an open channel 16 (FIG. 2) near one end of the inner wrap 14 that is configured to receive the other end of the inner wrap 14 to create a stabilizing effect such that the therapeutic wrap 10 may be securely positioned around the neck and/or other portion of the body. In some embodiments, the outer sleeve 12 may include one or more removable inserts 18 (FIG. 4) scented with a fragrance and/or essential oil to provide the additional benefit of aromatherapy.

Moving on to FIG. 2, shown is a drawing of the inner wrap 14 of the therapeutic wrap 10 according to various embodiments of the present disclosure. The inner wrap 14 comprises an elongated fabric extending from a first end to a second end. The inner wrap 14 further comprises a middle region 15 positioned between the first end and the second end. In some embodiments, the middle region 15 comprises a therapeutic insert 17 that comprises one or more sealed compartments 20a, 20b, 20c, 20d, 20e (FIG. 3, hereinafter “20”) each containing natural grain-like substances for providing therapeutic relief. In other embodiments, the one or more sealed compartments 20 are directly formed within the middle region 15 and not by the use of a therapeutic insert 17 as shown by the inner wrap 14b of the FIG. 10.

In one non-limiting example, the inner wrap 14 may comprise polyester and/or cotton fabrics. However, the elongated fabric may not be limited to these fabrics, and may comprise cotton, cotton sateen, linen, fleece, velour, sateen, bamboo, polyester, and/or any other appropriate type of fabric or combination thereof.

The inner wrap 14 may further comprise an open channel 16 near one end of the inner wrap 14. The open channel 16 may extend from a top portion to a bottom portion of the inner wrap 14. The open channel 16 is designed to receive the other end of the inner wrap 14 such that the other end of the inner wrap 14 may slide through the open channel 16 to create a stabilizing effect and minimize undesired movement of the therapeutic wrap 10 when positioned around the neck and/or other portion of a body. FIG. 1 illustrates this configuration of an end of the inner wrap 14 inserted through the open channel 16 of the inner wrap 14 such that the therapeutic wrap is securely positioned around the body.

Turning now to FIG. 3, shown is a drawing of the therapeutic insert 17 of the inner wrap 14 according to various embodiments of the present disclosure. Each of the one or more sealed compartments 20 is filled with a substantially equal distribution of natural substances. The compartment filing may comprise a combination of natural substances such as, for example, flaxseed, deer corn, rice, buckwheat, lavender buds and/or any other suitable natural substance. In one non-limiting example, each of the one or more compartments 20 may be filled with a combination of natural substances that may comprise about 10% flaxseed, about 88% rice, and about 2% lavender buds. However, this is merely an example on one embodiment of the combination of natural substances. In other embodiments, the type and/or amount of natural substances may vary as desired. The use of natural grain-like substances allows the therapeutic wrap 10 to anatomically conform to various body parts as needed.

Moving on to FIG. 4, shown is a drawing of an example of a front view of the outer sleeve 12 surrounding a portion of the inner wrap 14 according to various embodiments of the present disclosure. The outer sleeve 12 comprises a fabric, such as, for example, cotton, polyester, cotton sateen, velour, terry velour, fleece, linen, bamboo, and/or any other suitable fabric and/or combination thereof. In one non-limiting example, the outer portion of the outer sleeve 12 may comprise cotton and/or polyester, and the inner portion of the outer sleeve may comprise cotton and/or polyester. In a preferred embodiment, the outer sleeve 12 is washable and removable.

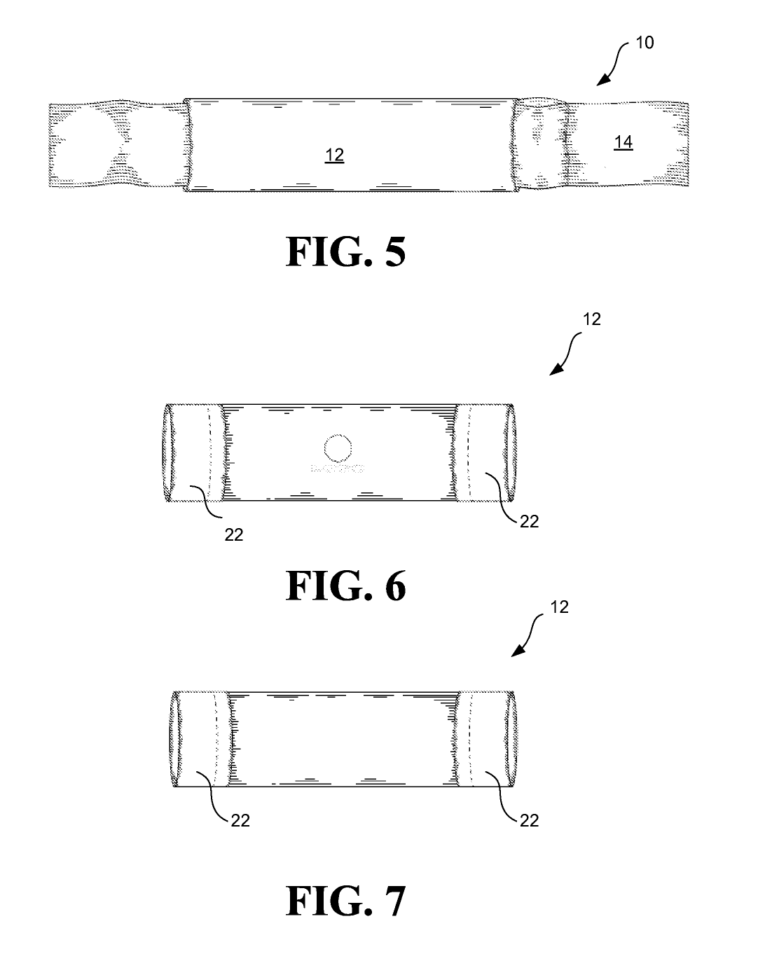

The outer sleeve 12 comprises one or more pockets 22a, 22b (hereinafter “22”) attached near each end of the outer sleeve 12. Each pocket 22 may be attached near a respective end of the outer sleeve 12 to allow for insertion of one or more removable inserts 18 that may be scented by a fragrance. In some embodiments, the one or more pockets 22 may be attached on the front of the outer sleeve 12 and not the back of the outer sleeve 12 as shown in FIGS. 4 and 5. In other embodiments, the one or more pockets 22 may surround a respective end of the front and back of the outer sleeve 12 to form a cuff-like configuration as shown in FIGS. 6 and 7. In such a configuration, the one or more removable inserts 18 may be placed in various positions of the one or more pockets 22 along the front side and/or back side of the outer sleeve 12. It should be noted that while the one or more pockets 22 are shown as being placed along the ends of the outer sleeve 12, other embodiments may include one or more pockets 22 attached to the outer sleeve 12 at other portions of the outer sleeve 12.

The one or more removable inserts 18 may comprise a cotton ball, cotton round, fabric, and/or other type of suitable material. The one or more removable inserts 18 may be scented with a preferred fragrance and/or essential oil to provide an additional benefit of aromatherapy to the therapeutic wrap 10 when in use. In one non-limiting example, one or more drops of essential/fragrant oil or other suitable type of fragrance may be added to the one or more removable inserts 18. Once scented with a preferred fragrance/essential oil, the one or more removable inserts 18 may be placed within a corresponding pocket 22 on the outer sleeve 12 such that the therapeutic wrap 10 provides aromatherapy in addition to therapeutic relief when in use. The one or more removable inserts 18 are removable to allow for the ability to use different fragrances as desired and/or add more fragrance as desired. In some embodiments, the one or more removable inserts 18 are disposable.

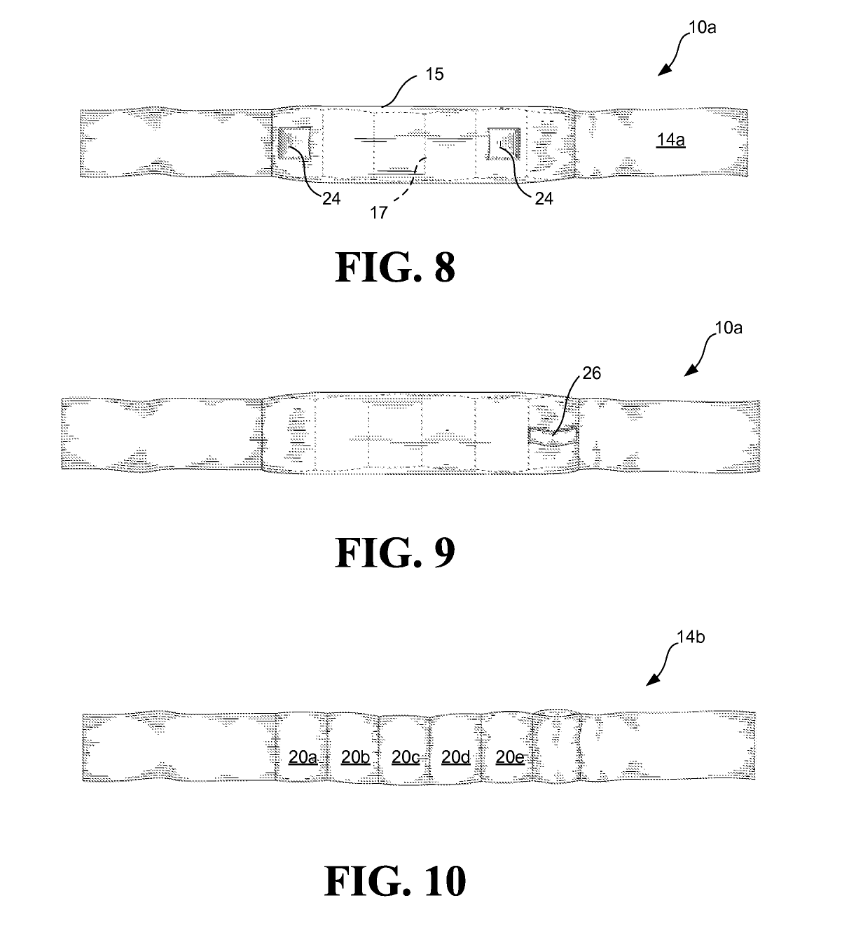

Turning now to FIG. 8, shown is a drawing of an example of a front view of another embodiment of the therapeutic wrap 10a. The therapeutic wrap 10a of FIG. 8 differs from the therapeutic wrap 10 of FIGS. 1, 4, and 5 as the therapeutic wrap 10a of FIG. 8 is a one-piece wrap capable of providing the benefit of aromatherapy without the use of an outer sleeve 12 (FIGS. 4-7).

The therapeutic wrap 10a of FIG. 8 comprises an inner wrap 14a similar to the inner wrap 14 of FIG. 2. In some embodiments, the therapeutic wrap 10a of FIG. 8 does not include the open channel 16. In other embodiments, the therapeutic wrap 10a may include the open channel 16. The inner wrap 14a of the therapeutic wrap 10a of FIG. 8 comprises a middle region 15 positioned between the first end and the second end of the therapeutic wrap 10a and one or more inner wrap pockets 24 that are designed to receive the one or more removable inserts 18. In some embodiments, the middle region 15 comprises a therapeutic insert 17 (FIG. 3) that comprises one or more sealed compartments 20 containing natural grain-like substances for providing therapeutic relief. In other embodiments, the one or more sealed compartments 20 are directly formed within the middle region 15 and not by the use of a therapeutic insert 17 (FIG. 10).

The one or more inner wrap pockets 22 are directly attached to the fabric of the inner wrap 14a of the therapeutic wrap 10a of FIG. 8. As such, one or more removable inserts 18 may be scented with a preferred scent, such as essential oils/fragrance oils, and inserted into the one or more inner wrap pockets 22. When the therapeutic wrap 10a is positioned about the body as desired, the user of the therapeutic wrap 10a may benefit from both therapeutic relief and aromatherapy.

Referring next to FIG. 9, shown is a back view of the therapeutic wrap 10a of FIG. 8, according to various embodiments of the present disclosure. The back of the therapeutic wrap 10a may comprise a band 26 extending along a portion of the therapeutic wrap 10a. The band 26 may comprise elastic, fabric, and or any other suitable material. The band 26 is sized to receive the other end of the inner wrap 14a such that the other end of the inner wrap 14a may slide through the band 26 to create a stabilizing effect and minimize undesired movement of the therapeutic wrap 10a when positioned around the neck and/or other portion of a body. The band 26 has a similar purpose as the open channel 16 discussed with respect to FIGS. 1, 2, 4, and 5.

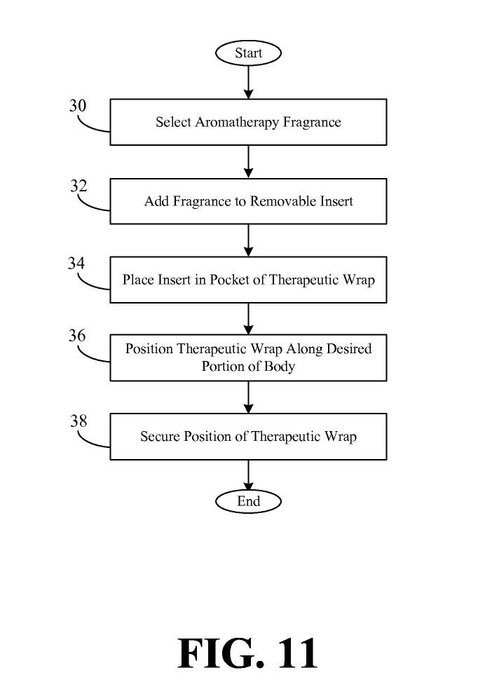

Moving on to FIG. 11, shown is a flowchart that provides one example of a method for using the therapeutic wrap 10, 10a. It is understood that the flowchart of FIG. 11 provides merely an example of the many different types of functional arrangements that may be employed to implement the operation of the methods as described herein.

At reference numeral 30, a preferred fragrance for aromatherapy is selected. At reference numeral 32, the preferred fragrance is added to one or more removable inserts 18. For example, a user may add one or more drops of essential/fragrant oil to the one or more removable inserts 18. At reference numeral 34, the one or more removable inserts 18 are inserted into the one or more pockets 22 on the therapeutic wrap 10, 10a. At reference numeral 36, the therapeutic wrap 10, 10a is positioned about portion of the body requiring therapeutic relief. For example, if a person desires relief around his or her neck, the therapeutic wrap 10, 10a may be positioned similarly to the position of the therapeutic wrap 10 shown in FIG. 1. At reference numeral 38, the therapeutic wrap is secured by placing the first end of the inner wrap 14, 14a, 14b through the open channel 16 (FIGS. 2, 4, and 5) or the band 26 (FIG. 9) disposed near the second end of the inner wrap 14, 14a, 14b.

Although the flowchart of FIG. 11 shows a specific order of execution, it is understood that the order of execution may differ from that which is depicted. For example, the order of execution of two or more steps may be scrambled relative to the order shown. Also, two or more steps shown in succession in FIG. 11 may be executed concurrently or with partial concurrence. Further, in some embodiments, one or more of the blocks shown in FIG. 11 may be skipped or omitted. It is understood that all such variations are within the scope of the present disclosure.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A therapeutic wrap, comprising:
an inner wrap extending from a first end to a second end and comprising one or more sealed chambers having a natural substance for providing therapeutic relief;
an outer sleeve surrounding a portion of the inner wrap;
a pocket attached to the outer sleeve; and
a scented insert inserted within the pocket, the scented insert being scented with at least one of an aromatherapy fragrance or essential oil.

2. The therapeutic wrap of claim 1, wherein the inner wrap further comprises an open channel near the second end of the inner wrap, the open channel extending from a top portion to a bottom portion of the inner wrap and being sized to allow for insertion of the first end of the inner wrap through the open channel.

3. The therapeutic wrap of claim 1, wherein the natural substance comprises at least one of the following: rice, corn, flaxseed, buckwheat or lavender buds.

4. The therapeutic wrap of claim 1, wherein the pocket is a cuff attached to the outer sleeve.

5. The therapeutic wrap of claim 1, wherein the outer sleeve and the inner wrap each comprise at least one of cotton or polyester.

6. The therapeutic wrap of claim 1, wherein the pocket is attached to the outer sleeve such that the pocket substantially surrounds the first end of the outer sleeve.

7. The therapeutic wrap of claim 1, wherein the pocket is attached to a front side of the outer sleeve.

8. The therapeutic wrap of claim 7, wherein the pocket is a first pocket, and the therapeutic wrap further comprises a second pocket attached to the second end of the front side of the outer sleeve.

9. The therapeutic wrap of claim 1, wherein the scented insert comprises at least one of a cotton ball or a cotton round.

10. The therapeutic wrap of claim 1, wherein the outer sleeve is washable and removable.

11. A therapeutic wrap, comprising:
an elongated fabric comprising a first end, a second end, and a middle region between the first end and the second end, the middle region including one or more sealed chambers filled with a natural substance mixture;
one or more pockets attached to the elongated fabric; and
one or more removable inserts inserted into the one or more pockets, the one or more removable inserts being scented with at least one of an aromatherapy essential oil or a fragrance oil.

12. The therapeutic wrap of claim 11, further comprising a channel extending from a top portion of the elongated fabric to a bottom portion of the elongated fabric, the channel being adjacent to the middle region.

13. The therapeutic wrap of claim 12, wherein the channel is positioned near a first end of the elongated fabric, and the channel is sized to receive a second end of the elongated fabric such that the second end can slide through the channel of the elongated fabric.

14. The therapeutic wrap of claim 11, wherein the natural substance mixture comprises at least one of: rice, corn, flaxseed, buckwheat, or lavender buds.

15. The therapeutic wrap of claim 13, wherein the elongated fabric further comprises at least one of polyester or cotton.

16. The therapeutic wrap of claim 13, wherein the removable insert is at least one of a cotton ball or cotton round.

17. A method, comprising:
adding an aromatherapy oil to one or more removable inserts;
inserting the one or more removable inserts into one or more pockets attached to a therapeutic wrap, wherein the therapeutic wrap comprises one or more natural substances used for therapeutic relief;
positioning the therapeutic wrap about a portion of a body; and
securing the therapeutic wrap about the portion of the body.

18. The therapeutic wrap of claim 17, wherein the aromatherapy oil is at least one of: an essential oil or a fragrance oil.

19. The therapeutic wrap of claim 17, wherein the one or more natural substances comprise at least one of: rice, corn, flaxseed, buckwheat, or lavender buds.

20. The therapeutic wrap of claim 17, wherein securing the therapeutic wrap about the portion of the body further comprises sliding a first end of the therapeutic wrap through a channel near the second end of the therapeutic wrap.
